(19) United States
(12) Patent Application Publication
(10) Pub. No.: US 2023/0390282 A1
(43) Pub. Date: Dec. 7, 2023

(54) TREATMENT OF EYE CONDITIONS

(71) Applicant: The University of Birmingham, Birmingham (GB)

(72) Inventors: Zubair Ahmed, Birmingham (GB); Richard Ian Tuxworth, Birmingham (GB)

(21) Appl. No.: 18/032,901

(22) PCT Filed: Oct. 15, 2021
(86) PCT No.: PCT/EP2021/078708

§ 371 Date: Apr. 20, 2023

(30) Foreign Application Priority Data
Oct. 21, 2020 (GB) 2016710.2

(51) Int. Cl. A61K 31/497; A61K 31/4184; A61P 27/02

(52) U.S. Cl. CPC A61K 31/497; A61K 31/4184; A61P 27/02

(57) ABSTRACT

The present disclosure relates to the treatment of eye conditions, by inhibiting Chk2 kinase. Particular ocular conditions may be associated with neuronal damage/dysfunction in the eye, or neurons in communication with the eye, which may result from, physical trauma, chemical means, infection, inflammation, hypoxia and/or interruption inblood supply, or be due to a neurodegenerative disorder and/or autoimmune disease. The Chk2 inhibitor may be a small molecule, protein, peptide or nucleic acid. Exemplary small molecule Chk2 inhibitors include PV1019, AZD7762, CCT241533, BML-277 or prexasertib.

### FIELD

The present disclosure relates to the treatment of eye conditions, by inhibiting Chk2 kinase.

### BACKGROUND

Double-strand breaks in DNA (DSBs) accumulate in neurons in many acute and chronic neurological conditions causing persistent activation of the DNA damage response (DDR), which leads to neural dysfunction, senescence and apoptosis (Simpson et al., 2015; Merlo et al., 2016 and Nagy et al., 1997) DSBs are sensed and processed by the MRN complex, comprising Mre11, Rad50 and NBS1/Nbn proteins (Lamarche et al., 2010), which recruits and activates the ataxia telangiectasia mutated (ATM) kinase or ataxia telangiectasia and Rad3-related (ATR) proteins. ATM is described as a DNA damage sensor and as a potential therapeutic target for treating cancer. ATM is a nodal point of the DNA damage response in cells and also interacts with many other proteins, including checkpoint-1 kinase (Chk1) and checkpoint-2 kinase (Chk2) in other pathways associated with cell-fate (Khalil et al, 2012).

The present disclosure is based on work conducted in relation to Chk2. Chk2 is a central multifunctional player in the induction of cell cycle arrest, DNA repair and apoptosis. The current understanding of Chk2 function in tumour cells, in both a biological and genetic context, suggests that inhibition of the kinase may be able to both sensitise tumour cells to certain damaging agents, whilst also protecting normal cells from damage, thus widening the therapeutic window. It has been demonstrated that disruption of the homologous recombination (HR) DNA repair pathway by Chk2 siRNA induces cellular sensitivity to the inhibition of poly (ADP-ribose) polymerase (PARP) activity. In addition, transgenic mouse studies have demonstrated that Chk2 abrogation gives rise to protection from radiation, raising the possibility that Chk2 inhibitors may be used as radioprotection agents.

### SUMMARY

The present disclosure is directed to work carried out by the inventors in relation to DNA damage respose in neurons and the role of Chk1 and Chk2. Surprisingly, the investigators found significant differences between inhibiting Chk1 and Chk2. These differences have lead to the targeting of Chk2 kinase as a means to prevent and/or treat or ameliorate ocular conditions associated with neuronal damage in the eye.

In a first aspect there is provided a Chk2 kinase inhibitor for use in a method of preventing or treating an ocular condition. Typically, the ocular condition is associated with neuronal damage or degeneration in the eye, or neurons in communication with the eye. In some embodiments the disclosure relates to protecting or treating neuronal damage or degeneration in the eye and/or neurons in communication with the eye. In some embodiments, the disclosure relates to neuronal regeneration of neurons in the eye and/or in communication with the eye. In some embodiments disclosure relates to treatment of the optic nerve and/or neurons in direct communication with the optic nerve.

Protecting against, treating neuronal damage or neuronal degeneration and/or promoting neuronal regeneration may include one or more of, protection of neural cells from apoptosis, promoting survival of neural cells, increasing the number of neural cell neurites, increasing neurite cell outgrowth, promoting retinal gliosis, promoting regeneration of neural cells and increasing or stimulation of neurotrophic factors in the nervous system.

In a further teaching, there is provided a method of treating a subject suffering from an ocular condition, which is associated with neuronal damage in the eye, or neurons in communication with the eye, the method comprising administering a Chk2 kinase inhibitor to an eye(s), or surrounding tissue, of the subject, in an amount sufficient to ameliorate or alleviate the condition.

A Chk2 kinase inhibitor (also referred to herein as Chk2 inhibitor), may be any suitable agent, which is capable of inhibiting Chk2 kinase, or inhibit expression of Chk2 kinase. Thus, the agent may be a molecule, such as a small chemical molecule (typically less than 500 Daltonsin size), which is capable of inhibiting Chk2 kinase or its expression in a cell, or may be a biological molecule, such as a protein, peptide, antibody (or active fragments thereof) or the like which is capable of inhibiting Chk2 kinase or its expression in a cell. For example a protein, peptide, antibody or antibody fragment may bind within the active site of Chk2 to prevent its activity, or act by preventing autophosphorylation and therefore activation of Chk2.

The term “inhibiting expression” is understood to include inhibition of transcription, inhibition of translation, enhanced degradation or reduced stability of a nucleic acid encoding Chk2 or the Chk2 protein itself. The term “inhibiting Chk2 kinase”, includes inhibition of phosphorylation as a means to inhibit activity, as well as inhibiting the binding of Chk2 kinase to a substrate, for example.

The Chk2 kinase inhibitor may also be a nucleic acid molecule, which is capable of inhibiting the expression of the Chk2 kinase gene, or a gene downstream of Chk2, but in the ATM-Chk2 pathway. Such downstream targets include p53, E2F1, Mdm2, BRCA1, cyclin dependent kinases. Such a molecule may include hydridising agents, such as antisense nucleic acid molecules (such as morpholino oligomers and phosphorodiamidate morphilino oligomers) , RNA interference using siRNA or shRNA for example, ribozymes, aptamers, CRISPR methods, TALENS and the like (see Joung & Sander (2013), Pickar-Oliver & Gersbach (2019) and Setten et al (2019), for example), which are well known to the skilled addressee and which are capable of binding to Chk2 nucleic acid (DNA or RNA), or nucleic acid which is upstream of the Chk2 gene and which are designed to prevent correct transcription and/or translation of nucleic acid encoding the Chk2 gene or its transcription product. Thus, any molecules which directly or indirectly reduce activity of Chk2 kinase in a cell or cells to be treated, as compared to Chk2 kinase activity within the cell or cells prior to administration of the Chk2 kinase inhibitor is envisaged for use in accordance with the disclosure.

In some embodiments of the present disclosure, Chk2 kinase inhibitors have a neuroprotective and/or neuroregenerative effect. In some embodiments, the Chk2 kinase inhibitors of the present disclosure have a neuroprotective and neuroregenerative effect. As the agents of the disclosure in certain embodiments have a neuroprotective effect, the agents may also be administered in advance or during surgery to the eye, in order to protect the eye from damage, which may occur as a result of surgery, to the eye or tissue/nerves associated with the eye. Thus, the present disclosure also extends to prophylactic uses of the Chk2 inhibitors in a subject.

A Chk2 kinase inhibitor for use in accordance with the present disclosure may also inhibit another molecule(s). For example, in one embodiment, a suitable Chk2 inhibitor may also inhibit Chk1 kinase. However, in some embodiments the molecules may be more selective for inhibiting Chk2 kinase than another molecule/kinase/enzyme, such as Chk1. Thus, in one embodiment the Chk2 inhibitor may be at least 2-fold, 4-fold, 10-fold, or 25-fold more selective for Chk2 kinase, than another molecule/kinase/enzyme, such as Chk1. However, in some embodiments the Chk2 inhibitor may be equally or less selective for inhibiting another molecule/kinase/enzyme, such as Chk1.

Exemplary Chk2 inhibitory molecules suitable for use in accordance with the present disclosure are described, for example, in (Jobson et al., 2009; Zabludoff et al., 2008; Anderson et al., 2011; Arienti et al., 2005; King et al., 2015). In some embodiments the Chk2 inhibitor is Prexasertib (ID₅₀=8 nM), BML-227 (IC₅₀=15 nM), CCT241533 (IC₅₀=3 nM), or AZD-7762 (IC₅₀=5 nM).

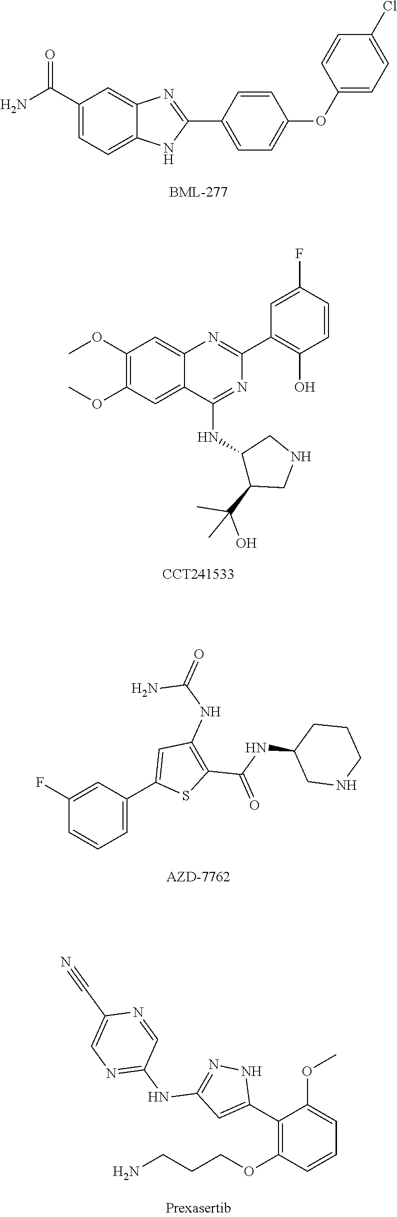 [SMILES: NC(=O)c1ccc2[nH]c(-c3ccc(Oc4ccc(Cl)cc4)cc3)nc2c1 ; COc1cc2nc(-c3cc(F)ccc3O)nc(N[C@@H]3CNC[C@H]3C(C)(C)O)c2cc1OC ; NC(=O)Nc1cc(-c2cccc(F)c2)sc1C(=O)N[C@H]1CCCNC1 ; COc1cccc(OCCCN)c1-c1cc(Nc2cnc(C#N)cn2)n[nH]1]

As mentioned above, the present disclosure is directed to preventing or treating occular conditions, which are associated with neuronal dysfunction and/or damage (including damage to DNA damage), such as caused by trauma, neural degeneration, pressure within the eye, inflammation, infection and interruption in blood supply to the eye, for example. Neuronal damage may occur to any neurons within the eye, including the optic nerve and/or neurons which directly communicate with neurons within the eye and/or optic nerve.

The ocular condition may be sporadic and/or inherited.

The ocular condition may result from neuronal damage. The neuronal damage may be caused, for example, by physical means and/or by chemical means. The physical means may result from, for example, surgery or trauma. Types of trauma may include, for example, blunt force, penetration, compression, pressure, and/or blast trauma. The surgery may be resection, decompression or reparative surgery, for example. The chemical means may be a drug, neurotoxin, infection, inflammation, autoimmune disease, oxidative stress, nitrosative stress.

The occular condition may be a neurodegenerative condition, such as age-related macular degeneration, glaucoma, diabetic retonopathy or neurodegenerative diseases that also affect the eye including Alzheimer's and Parkinson's Disease.

The ocular condition may result from blood flow damage/disruption. The blood flow damage/disruption may temporary or permanent and/or be caused by, for example, stroke, ischaemia, re-oxygenation of tissues, vascular disorder, transient ischemic attack (TIA), hydrocephalus, hemorrhage/hematoma. The ocular condition may result from damage to blood vessels, which may be as a consequence of diabetes or premature birth, for example.

The occular condition may be as the result of an infection. This may be caused by a bacterial, viral, parasitic, fungal and/or mycobacterial infection. The infection may be for example caused by measles, herpes, polio, zika, coronavirus, meningococcus, or plasmodium.

Where the neuronal damage is due to trauma, this includes physical trauma as caused by a subject receiving physical damage to the eye due to an external force, or material penetrating the eye, as well as physical trauma to the head in general, which can further lead to associated problems in the eye. Additional traumatic conditions associated with the eye include retinal ischemia, acute retinopathy associated with trauma, postoperative complications, traumatic optic neuropathy (TON); and damage related to laser therapy (including photodynamic therapy (PDT)), damage related to surgical light-induced iatrogenic retinopathy, and damage related to corneal transplantation and stem cell transplantation of ocular cells.

Traumatic optic neuropathy refers to acute damage of the optic nerve secondary to trauma of the eye in general. Optic nerve axons can be directly or indirectly damaged, and vision loss can be partial or complete. Indirect damage to the optic nerve is typically caused by a force transfer from blunt head trauma to the nerve cervical canal. This is in contrast to direct TON resulting from anatomical destruction of optic nerve fibers from penetrating orbital trauma, bone fragments within the neural transluminal tube, or schwannoma. Patients who have received corneal transplants or ocular stem cell transplants can also suffer trauma.

As well as neural damage caused by trauma, other conditions which may be treated in accordance with the present invention include, optic neuritis, glaucoma, and neurodegenerative conditions in general where damage to neurons within the eye are an associated or secondary issue.

Optic neuritis occurs when swelling (inflammation) damages the optic nerve. Common symptoms of optic neuritis include pain with eye movement and temporary vision loss in one eye. Signs and symptoms of optic neuritis can be the first indication of multiple sclerosis (MS), or they can occur later in the course of MS. Multiple sclerosis is a disease that causes inflammation and damage to nerves in your brain as well as the optic nerve. Thus, in one embodiment, the present disclosure includes the treatment of eye damage caused by a subject suffering from MS.

Besides MS, optic nerve inflammation can occur with other conditions, including infections or immune diseases, such as lupus. Another disease called neuromyelitis optica causes inflammation of the optic nerve and spinal cord.

Glaucoma can be divided into approximately two main categories: “open angle” or chronic glaucoma and “closed angle” or acute glaucoma. Angle-closure acute glaucoma appears suddenly, often with painful side effects, and is usually diagnosed quickly, but damage and loss of vision can also occur very suddenly. Primary open-angle glaucoma (POAG) is a progressive disease that results in optic nerve damage and ultimately loss of vision. Glaucoma causes neurodegeneration of the retina and optic disc. Even with aggressive medical care and surgical procedures, the disease generally persists, with gradual loss of retinal neurons, decreased visual function, and ultimately blindness. Treatment of open angle and closed angle glaucoma is envisaged in accordance with the present disclosure.

Additionally, subjects with neurodegenerative conditions including Parkinson's disease (PD); Alzheimer's disease (AD); amyotrophic lateral sclerosis (ALS); motor neuron disease (MND); and Huntington's disease (HD), may suffer from eye problems associated with neurodegeneration within the eye. Other inherited conditions include neuronal ceroid lipofuscinoses (NCLs) and related lysosomal storage disorders, where progressive optic atrophy occurs early in the disease course. The present disclosure includes treatment of such eye problems associated with such neurodegenerative conditions.

The Chk2 kinase inhibitor may be the only active agent, which is administered to the subject, or may be administered in combination with one or more active agents, which are not Chk2 inhibitors. In one embodiment the other agent is an inhibitor of another enzyme, such as a PARP and/or Chk1 inhibitor, a matrix metalloprotease (see for example WO2017199042) and/or a water channel protein such as aquaporin 4 (see Kitchen et al., 2020, Cell 181: 784-799). An “active agent” means a compound (including a compound disclosed herein), element, or mixture that when administered to a patient, alone or in combination with another compound, element, or mixture, confers, directly or indirectly, a physiological effect on the subject. The indirect physiological effect may occur via a metabolite or other indirect mechanism.

The combination of the agents listed above with a compound of the present invention would be at the discretion of the physician who would select dosages using his common general knowledge and dosing regimens known to a skilled practitioner.

Where a compound of the invention is administered in combination therapy with one, two, three, four or more, preferably one or two, preferably one other therapeutic agents, the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer period apart where required), the precise dosage regimen being commensurate with the properties of the therapeutic agent(s).

The compounds of the invention may also be administered in conjunction with non-active agent treatments such as, photodynamic therapy, gene therapy; surgery.

The subject is typically an animal, e.g. a mammal, especially a human.

By a therapeutically or prophylactically effective amount is meant one capable of achieving the desired response, and will be adjudged, typically, by a medical practitioner. The amount required will depend upon one or more of at least the active compound(s) concerned, the patient, the condition it is desired to treat or prevent and the formulation of order of from 1 μg to 1 g of compound per kg of body weight of the patient being treated.

Different dosing regimens may likewise be administered, again typically at the discretion of the medical practitioner. Compounds of the disclosure, may be provided by daily administration although regimes where the compound(s) is (or are) administered more infrequently, e.g. every other day, weekly or fortnightly, for example, are also embraced by the present disclosure.

By treatment is meant herein at least an amelioration of a condition suffered by a patient; the treatment need not be curative (i.e. resulting in obviation of the condition). Analogously references herein to prevention or prophylaxis herein do not indicate or require complete prevention of a condition; its manifestation may instead be reduced or delayed via prophylaxis or prevention according to the present disclosure.

The compounds for use in methods according to the present disclosure, may be provided as the compound itself or a physiologically acceptable salt, solvate, ester or other physiologically acceptable functional derivative thereof. These may be presented as a pharmaceutical formulation, comprising the compound or physiologically acceptable salt, ester or other physiologically functional derivative thereof, together with one or more pharmaceutically acceptable carriers therefor and optionally other therapeutic and/or prophylactic ingredients.

Any carrier(s) are acceptable in the sense of being compatible with the other ingredients of the formulation and not deleterious to the recipient thereof.

Examples of physiologically acceptable salts of the compounds according to the disclosure include acid addition salts formed with organic carboxylic acids such as acetic, lactic, tartaric, maleic, citric, pyruvic, oxalic, fumaric, oxaloacetic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids.

Physiologically functional derivatives of compounds of the present disclosure are derivatives, which can be converted in the body into the parent compound. Such physiologically functional derivatives may also be referred to as “pro-drugs” or “bioprecursors”. Physiologically functional derivatives of compounds of the present disclosure include hydrolysable esters or amides, particularly esters, in vivo. Determination of suitable physiologically acceptable esters and amides is well within the skills of those skilled in the art.

It may be convenient or desirable to prepare, purify, and/or handle a corresponding solvate of the compounds described herein, which may be used in the any one of the uses/methods described. The term solvate is used herein to refer to a complex of solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

It will be appreciated that the compounds of the present disclosure may exist in various stereoisomeric forms and the compounds of the present disclosure as hereinbefore defined include all stereoisomeric forms and mixtures thereof, including enantiomers and racemic mixtures. The present disclosure includes within its scope the use of any such stereoisomeric form or mixture of stereoisomers, including the individual enantiomers of the compounds of formulae (I) or (II) as well as wholly or partially racemic mixtures of such enantiomers.

The compounds of the present disclosure may be purchased from commercial suppliers, or prepared using reagents and techniques readily available in the art.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. Methods typically include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersible granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active compound is formulated in an appropriate release-controlling matrix, or is coated with a suitable release-controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds.

Pharmaceutical formulations suitable for parenteral administration include sterile solutions or suspensions of an active compound in aqueous or oleaginous vehicles.

Injectible preparations may be adapted for bolus injection or continuous infusion. Such preparations are conveniently presented in unit dose or multi-dose containers, which are sealed after introduction of the formulation until required for use. Alternatively, an active compound may be in powder form, which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility, an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

It should be understood that in addition to the aforementioned carrier ingredients the pharmaceutical formulations described above may include, an appropriate one or more additional carrier ingredients such as diluents, buffers, flavouring agents, binders, surface active agents, thickeners, lubricants, preservatives (including anti-oxidants) and the like, and substances included for the purpose of rendering the formulation isotonic with the blood of the intended recipient.

Pharmaceutically acceptable carriers are well known to those skilled in the art and include, but are not limited to, 0.1 M and preferably 0.05 M phosphate buffer or 0.8% saline. Additionally, pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

In some embodiments, pharmaceutical formulations of the invention are particularly suited for ophthalmic administration, which is directly administered to the eye.

In some embodiments, such ophthalmic formulations may be administered topically with eye drops. In other embodiments, the ophthalmic formulations may be administered as an irrigating solution. In other embodiments, the ophthalmic formulations may be administered periocularly. In other embodiments, the ophthalmic formulations may be administered intraocularly.

In another teaching, the disclosure provides a topical, periocular, or intraocular ophthalmic formulation useful for neuroprotection and/or neuroregeneration in a subject suffering from or at risk of ocular impairment or vision loss due to neural damage.

Topical ophthalmic formulations administered in accordance with the present disclosure may also include various other ingredients including, but not limited to, surfactants, tonicity agents, buffers, preservatives, cosolvents, and thickeners.

A topical ophthalmic formulation administered topically, periocularly or intraocularly comprises an ophthalmically effective amount of one or more Chk2 inhibitors as described herein. As used herein, an “ophthalmically effective amount” is an amount sufficient to reduce or eliminate the signs or symptoms of an ocular condition described herein. In general, for formulations intended for topical administration to the eye in the form of eye drops or eye ointments, the total amount of active agent may be 0.001 to 1.0% (w/w). When applied as eye drops, 1-2 drops (approximately 20-45 μl each) of such formulations may be administered once to several times a day.

Chk2 inhibitors of the present disclosure may be conjugated to a cell penetrating peptide, for example, to aid with delivery of the Chk2 inhibitor to the eye

One route of administration is local. The compounds of the present disclosure can be administered as solutions, suspensions, or emulsions (dispersants) in an ophthalmically acceptable vehicle. An “ophthalmically acceptable” component, as used herein, refers to a component that does not cause any significant eye damage or discomfort over the intended concentration and intended use time. Solubilizers and stabilizers should be non-reactive. “Ophthalmically acceptable vehicle” refers to any substance or combination of substances that is non-reactive with the compound and suitable for administration to a patient. Suitable vehicles include physiologically acceptable oils such as silicone oil, USP mineral oil, white oil, poly (ethylene-glycol), polyethoxylated castor oil and vegetable oils such as corn oil or peanut oil Can be a non-aqueous liquid medium. Other suitable vehicles may be aqueous or oil-in-water solutions suitable for topical application to the patient's eye. These vehicles can preferably be based on ease of formulation and the ease with which a patient can administer such formulations due to the instillation of 1-2 drops of solution onto the affected eye. Formulations can also be suspensions, viscous or semi-viscous gels, or other types of solid or semi-solid formulations, and fatty bases (natural waxes such as beeswax, carnauba wax, wool wax (wool oil) (Wool fat)), refined lanolin, anhydrous lanolin); petroleum wax (eg, solid paraffin, microcrystalline wax); hydrocarbon (eg, liquid paraffin, white petrolatum, yellow petrolatum); or combinations thereof). The formulation can be applied manually or by use of an applicator (such as a wipe, contact lens, dropper, or spray).

Various tonicity agents can be used to adjust the tonicity of the composition, preferably to that of natural tears for ophthalmic compositions. For example, sodium chloride, potassium chloride, magnesium chloride, calcium chloride, dextrose, and/or mannitol can be added to the composition to approximate physiological tonicity. The amount of such isotonic agent will vary depending on the particular agent to be added. In general, however, the formulation will have a sufficient amount of tonicity agent so that the final composition has an osmolality that is ophthalmically acceptable (generally about 200-400 mOsm/kg).

Other agents may also be added to the topical ophthalmic formulation of the present disclosure to increase the viscosity of the carrier. Examples of viscosity enhancing agents include, but are not limited to: polysaccharides (such as hyaluronic acid and its salts, chondroitin sulfate and its salts, dextran, polymers of various cellulose families); vinyl polymers; and acrylics Acid polymer. In general, a phospholipid carrier or artificial tear carrier composition exhibits a viscosity of 1 to 400 centipoise.

An appropriate buffer system (eg, sodium phosphate, sodium acetate, sodium citrate, sodium borate, or boric acid) can be added to the formulation to prevent pH fluctuations under storage conditions. The specific concentration will vary depending on the agent used. However, preferably the buffer is selected to maintain a target pH within the range of pH 6 to 7.5.

Formulations of the disclosure may be administered intraocularly after a traumatic event involving retinal tissue and optic nerve head tissue or before or during ophthalmic surgery to prevent injury or damage. Formulations useful for intraocular administration are generally intraocular injection formulations or surgical washes.

Compounds and formulation of the present disclosure may also be administered by periocular or intraocular administration and can be formulated in a solution or suspension for periocular/intracocular administration. The compounds/formulations of the disclosure may be administered periocularly/intraocularly after traumatic events involving retinal tissue and optic nerve head tissue or before or during ophthalmic surgery to prevent injury or damage.

Formulations useful for periocular/intracocular administration are generally in the form of injection formulations or surgical lavage fluids.

Periocular administration refers to administration to tissues near the eye (such as administration to tissues or spaces around the eyeball and in the orbit). Periocular administration can be performed by injection, deposition, or any other mode of placement. Periocular routes of administration include, but are not limited to, subconjunctival, suprachoroidal, near sclera, near sclera, subtenon, subtenon posterior, retrobulbar, periocular, or extraocular delivery. Intraocular delivery refers to administration directly into the eye, such as by way of injection, or by way of a depot surgically inserted into the eye, for example.

Therapeutic formulations for veterinary use may be in any of the above-mentioned forms, but conveniently may be in either powder or liquid concentrate form. In accordance with standard veterinary formulation practice, conventional water-soluble excipients, such as lactose or sucrose, may be incorporated in the powders to improve their physical properties. Thus, particularly suitable powders of this invention comprise 50 to 100% w/w and preferably 60 to 80% w/w of the active ingredient(s) and 0 to 50% w/w and preferably 20 to 40% w/w of conventional veterinary excipients. These powders may either be added to animal feedstuffs, for example by way of an intermediate premix, or diluted in animal drinking water.

Liquid concentrates of this invention suitably contain the compound or a derivative or salt thereof and may optionally include a veterinarily acceptable water-miscible solvent, for example, polyethylene glycol, propylene glycol, glycerol, glycerol formal or such a solvent mixed with up to 30% v/v of ethanol. The liquid concentrates may be administered to the drinking water of animals.

### DETAILED DESCRIPTION

The present disclosure will now be further described by way of example and with reference to the Figures, which show:

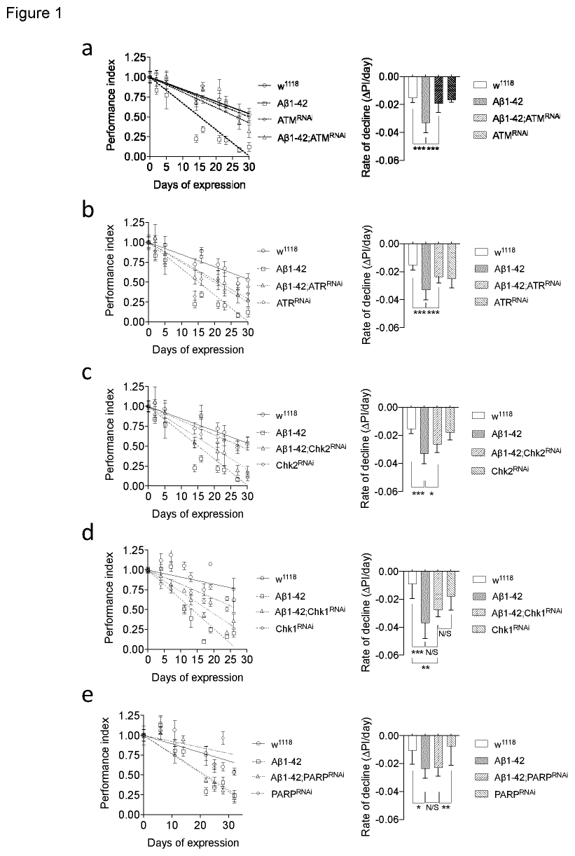

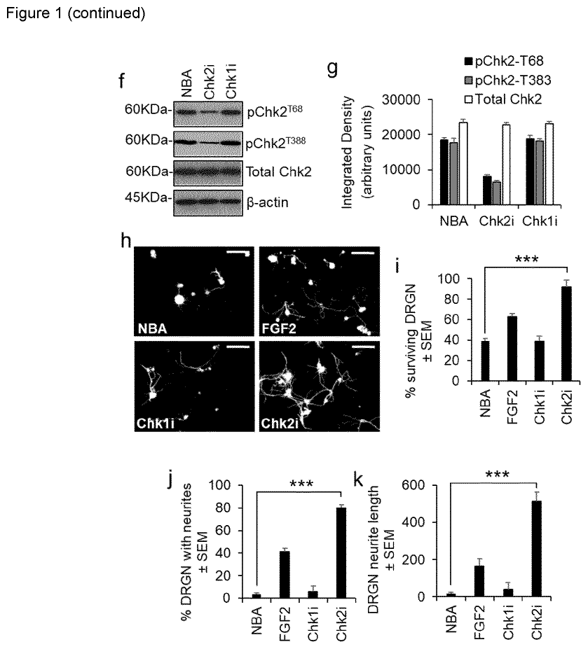

FIG. 1. Inhibition of Chk2 maintains neural function in an amyloid toxicity model in Drosophila and promotes neuroprotection and neurite outgrowth in DRGN cultures. a-e. Longitudinal startle responses of Drosophila expressing amyloid beta (Aβ₁₋₄₂) in adult neurons. Knockdown by RNAi of a. ATM (tefu); b. ATR (mei-41); c. Chk2 (lok) and d. Chk1 (grp) all significantly slow the rate of decline of the startle response induced by Aβ₁₋₄₂. e. Knockdown of Parp has no effect. ***P=0.0001, *P=0.05, ANOVA with Dunnett's post hoc test. n=5 for all genotypes. f Western blot and g densitometry to show that Chk2i suppresses pChk2 T68 and pChk2 T383 in DRGN cultures. h Representative images after treatment with Chk2i and quantification to show that Chk2i enhances i % surviving DRGN j % DRGN with neurites and k the mean neurite length. ***P=0.0001, ANOVA with Dunnett's post hoc test. n=3 wells/treatment, 3 independent repeats (total n=9 wells/treatment). Scale bars in h=50 μm;

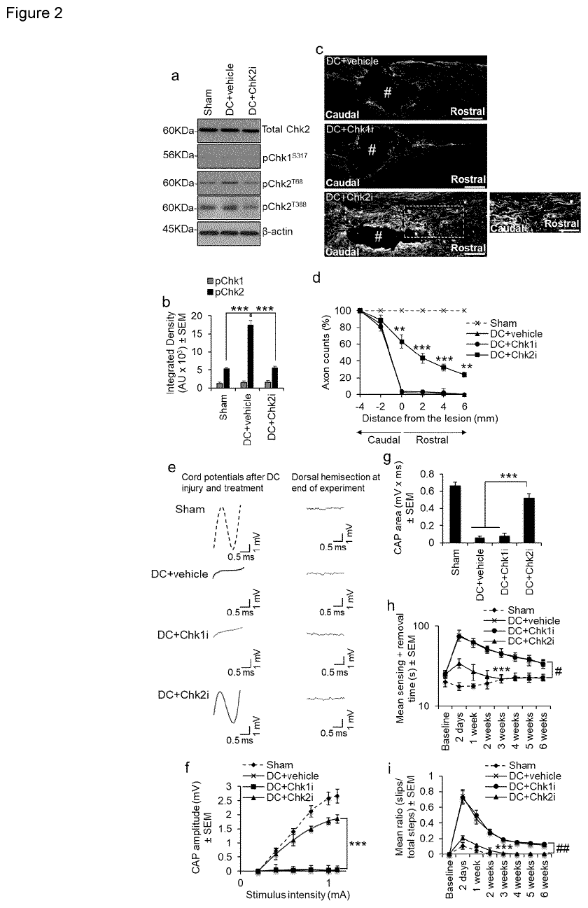

FIG. 2. Inhibition of Chk2 promotes Dorsal Column (DC) axon regeneration in vivo. a Western blot and b densitometry to show that Chk2i significantly suppresses pChk2^{T68} and pChk2^{T383} levels after DC injury without affecting pChk1 levels. c Many GAP43⁺ axons (green

(DAPI⁺ nuclei=blue)) were observed in DC+Chk2i regenerating through the lesion site and into the rostral cord (boxed region=high power view of GAP43⁺ axons in the rostral cord) despite the presence of a large cavity (#), whilst few GAP43⁺ axons were present beyond the lesion site in DC+vehicle and DC+Chk1i-treated spinal cords. d Quantification of the number of GAP43⁺ axons at distances caudal and rostral to the lesion site showing significant proportions of axons regenerating up to 6mm beyond the lesion epicentre. Scale bars in c=200 μm. **P=0.0012; ***P=0.0001, ANOVA with Dunnett's post hoc test. n=6 nerves/treatment, 3 independent repeats (total n=18 nerves/treatment). e Spike 2 software-processed CAP traces from representative sham controls, DC+vehicle, DC+Chk1i and DC+Chk2i-treated rats at 6 weeks after DC injury and treatment. Dorsal hemisection at the end of recording ablated all CAP traces. f Negative CAP amplitudes and g CAP area at different stimulation intensities were both significantly attenuated in DC+vehicle- and DC+Chk1i-treated rats but were restored in DC+Chk2i-treated rats (P=0.0001, one-way ANOVA with Dunnett's post hoc test (main effect)). h Mean tape sensing/removal times and I mean error ratio to show the number of slips vs total steps are both restored to normal 3 weeks after treatment with Chk2i (***P=0.0001, independent sample t-test (DC+vehicle vs. DC+Chk2i at 3 weeks) whilst a significant deficit remains in DC+vehicle- and DC+Chk1i-treated rats (#=P=0.00014, generalized linear mixed models and ##=P=0.00011, linear mixed models over the whole 6 weeks). n=6 rats/treatment, 3 independent repeats (total n=18 rats/treatment);

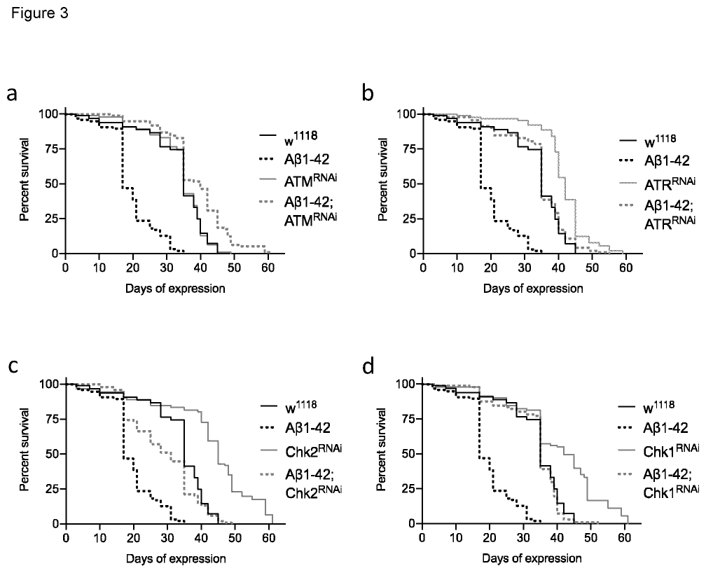

FIG. 3. Knockdown of ATM, Chk2, ATR or Chk1 extends the lifespan of Aβ₁₋₄₂ expressing Drosophila. Kaplan-Meier survival of adult Drosophila expressing a secreted form of human Aβ₁₋₄₂ in neurons under the control of Elav-Gal4. Expression was restricted to adult neurons by use of the Gal80^{ts} system. Flies were developed at the restrictive temperature of 18° C. to prevent expression and shifted to a permissive temperature of 27° C. on the day of eclosion. Survival was assessed 2-3 times per week. Aβ₁₋₄₂ vs. Aβ₁₋₄₂; UAS-RNAi flies were compared by Log-Rank analysis in GraphPad Prism 8;

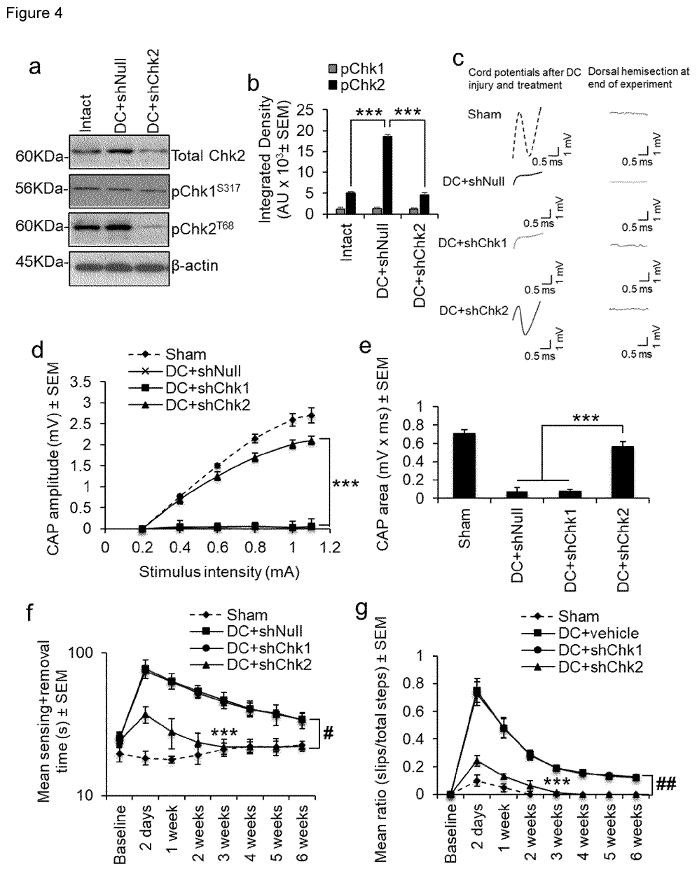

FIG. 4. Inhibition of Chk2 using BML-277 promotes significant functional recovery after DC injury in vivo. a Western blot and densitometry to show that 5 μg of BML-277 optimally suppresses pChk2^{T68} after DC injury. b Spike 2 software-processed CAP traces at 6 weeks after DC injury from representative Sham controls, DC+vehicle, DC+Chk1i and DC+BML-277-treated rats. Dorsal hemisection at the end of recording ablated all CAP traces. c Negative CAP amplitudes were significantly attenuated in DC+vehicle- and DC+Chk1i-treated rats but were restored in DC+ML-277-treated rats (P=0.0001, one-way ANOVA with Dunnett's post hoc test (main effect)). d Mean CAP area at different stimulation intensities were significantly attenuated in DC+vehicle- and DC+Chk1i-treated rats but improved significantly in DC+BML-277-treated rats (P=0.0001, one-way ANOVA with Dunnett's post hoc test (main effect)). e Mean tape sensing and removal times were restored to normal 4 weeks after treatment with BML-277 (P=0.0001, independent sample t-test (DC+vehicle vs. DC+BML-277 at 4 weeks) whilst a significant deficit remained in DC+vehicle- and DC+Chk1i-treated rats (#=P=0.00013, generalized linear mixed models over the whole 6 weeks). f Mean error ratio to show the number of slips vs total number of steps in the horizontal ladder walking test also returns to normal 4 weeks after treatment with shChk2 (P<0.00011, independent sample t-test (DC+vehicle vs DC+BML-277 at 4 weeks)), with a deficit remaining in DC+vehicle- and DC+Chk1i-treated rats (#=P=0.00011, linear mixed models over the whole 6 weeks). n=6 rats/treatment, 3 independent repeats (total n=18 rats/treatment).;

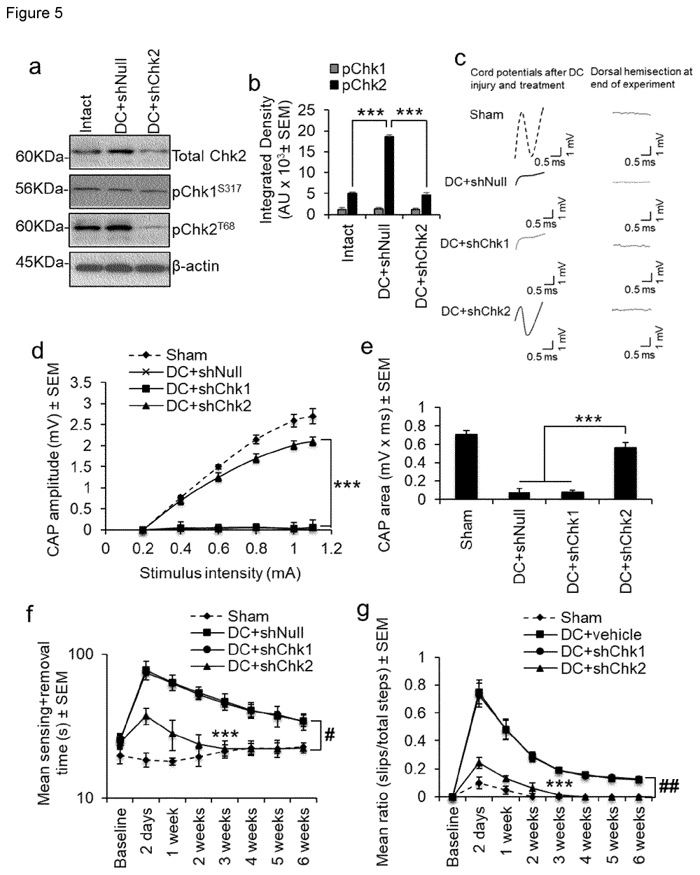

FIG. 5. Inhibition of Chk2 using non-viral plasmid DNA and delivered using in vivo-JetPEI (PEI) promotes significant functional repair after DC injury in vivo. a and b PEI delivered plasmids significantly suppress pChk2^{T68} and pChk2^{T383} levels in spinal L4/L5 DRGs at 4 weeks after DC injury without affecting pChk1 levels. n=12 DRG/treatment (6 rats/treatment), 3 independent repeats (total n=36 DRG/treatment (18 rats/treatment)). c Spike 2 software-processed CAP traces at 6 weeks after DC injury from representative Sham controls, DC+shNull, DC+shChk1i and DC+shChk2i-treated rats. Dorsal hemisection at the end of the experiment ablates CAP traces. d Negative CAP amplitudes were significantly attenuated in DC+shNull- and DC+shChk1-treated rats but were restored in DC+shChk2-treated rats (P=0.0001, one-way ANOVA with Dunnett's post hoc test (main effect)). e Mean CAP area at different stimulation intensities were significantly attenuated in DC+shNull- and DC+shChk1-treated rats but improved significantly in DC+shChk2-treated rats (P=0.0001, one-way ANOVA Dunnett's post hoc test (main effect)). f Mean tape sensing and removal times were restored to normal 3 weeks after treatment with shChk2 (P=0.0001, independent sample t-test (DC+shNull vs. DC+shChk2 at 3 weeks) whilst a significant deficit remained in DC+shNull- and DC+shChk1-treated rats (#=P=0.00013, generalized linear mixed models over the whole 6 weeks). g Mean error ratio to show the number of slips vs total number of steps in the horizontal ladder walking test also returns to normal 3 weeks after treatment with shChk2 (P=0.00011, independent sample t-test (DC+shNull vs DC+shChk2 at 3 weeks)), with a deficit remaining in DC+shNull- and DC+shChk1-treated rats (#=P=0.0001, linear mixed models over the whole 6 weeks). n=6 rats/treatment, 3 independent repeats (total n=18 rats/treatment);

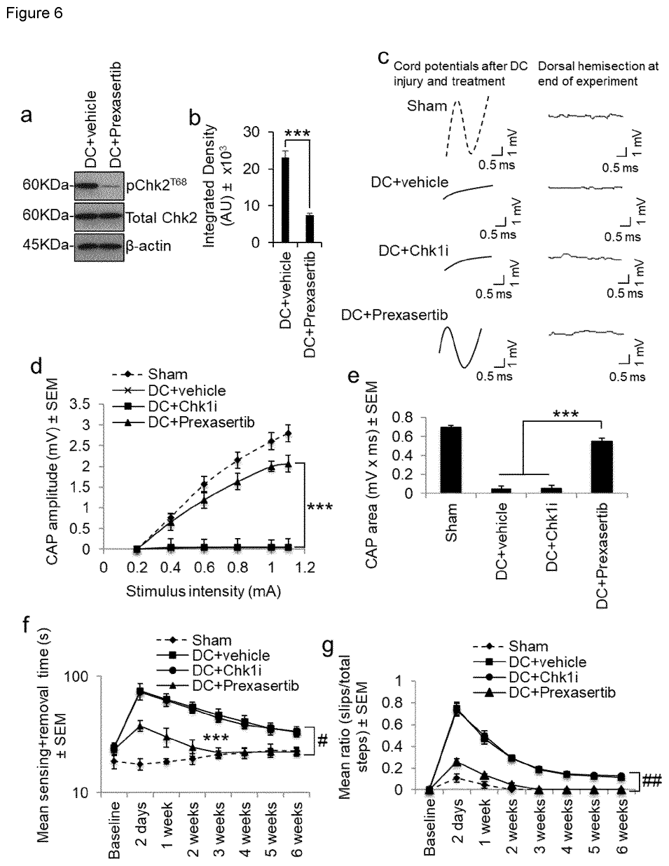

FIG. 6. Inhibition of Chk2 using Prexasertib promotes significant functional recovery after DC injury in vivo. a Western blot and b densitometry to show that 3 μg of Prexasertib optimally suppresses pChk2^{T68} after DC injury. c Spike 2 software-processed CAP traces at 6 weeks after DC injury from representative Sham controls, DC+vehicle, DC+Chk1i and DC+Prexasertib-treated rats. Dorsal hemisection at the end of recording ablated all CAP traces. d Negative CAP amplitudes were significantly attenuated in DC+vehicle- and DC+Chk1i-treated rats but were restored in DC+Prexasertib-treated rats (P=0.0001, one-way ANOVA with Dunnett's post hoc test (main effect)). e Mean CAP area at different stimulation intensities were significantly attenuated in DC+vehicle- and DC+Chk1i-treated rats but improved significantly in DC+Prexasertib-treated rats (P=0.0001, one-way ANOVA with Dunnett's post hoc test (main effect)). f Mean tape sensing and removal times were restored to normal 3 weeks after treatment with Prexasertib (P=0.0001, independent sample t-test (DC+vehicle vs. DC+Prexasertib at 3 weeks) whilst a significant deficit remained in DC+vehicle- and DC+Chk1i-treated rats (#=P=0.00014, generalized linear mixed models over the whole 6 weeks). g Mean error ratio to show the number of slips vs total number of steps in the horizontal ladder walking test also returns to normal 4 weeks after treatment with shChk2 (P<0.00014, independent sample t-test (DC+vehicle vs DC+Prexasertib at 3 weeks)), with a deficit remaining in DC+vehicle- and DC+Chk1i-treated rats (##=P=0.00012, linear mixed models over the whole 6 weeks). n=6 rats/treatment, 3 independent repeats (total n=18 rats/treatment);

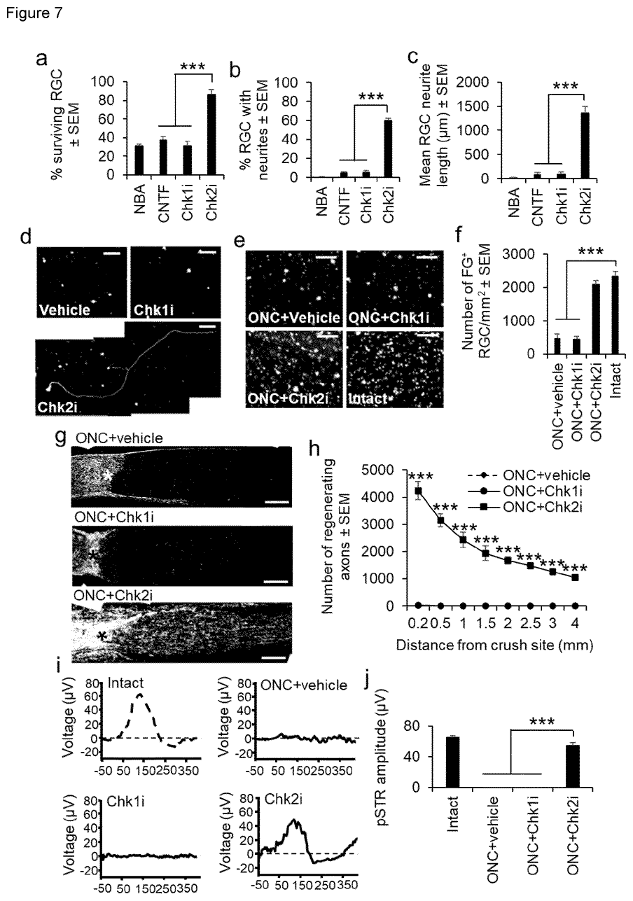

FIG. 7. Chk2 inhibition prevents RGC apoptosis and stimulates neurite outgrowth/axon regeneration after 4 days in vitro and 24d after ONC in vivo. a Pre-optimised Chk2i concentration in culture at 4 days significantly enhanced RGC survival compared to control NBA, positive control CNTF (preoptimized) or Chk1i. b Chk2i also enhanced the % RGC with neurites and the c mean neurite length compared to all other treatment groups. d Representative images from RGC treated with vehicle, Chk1i and Chk2i. n=3 wells/treatment, 3 independent repeats (total n=9 wells/treatment). e Representative images of FG-labelled RGC in retinal wholemounts at 24 days after ONC in vivo and f quantification to show that Chk2i significantly neuroprotected RGC from death. g

Representative images of longitudinal sections of optic nerves at 24 days after ONC stained for GAP43 from ONC+vehicle, ONC+Chk1i and ONC+Chk2i and h, quantification to show that Chk2i significantly enhanced RGC axon regeneration through the lesion site (*) and into the distal optic nerve segment (n=6 nerves/condition, 3 independent repeats (total n=18 nerves/condition). ***P=0.0001, ANOVA with Dunnett's post hoc test. Scale bars in g=200 μm. i Representative ERG traces and j Photopic scotopic threshold (pSTR) amplitude quantification from Intact, ONC+vehicle, ONC+Chk1i and ONC+Chk2i-treated rats to show preservation of a significant ERG trace and pSTR amplitude after Chk2i, which is normally ablated in ONC+vehicle treatment. Chk1i had no effect on ERG traces. ***P=0.0001, ANOVA with Dunnett's post hoc test. n=6 eyes/treatment, 3 independent repeats, total n=18 eyes/treatment;

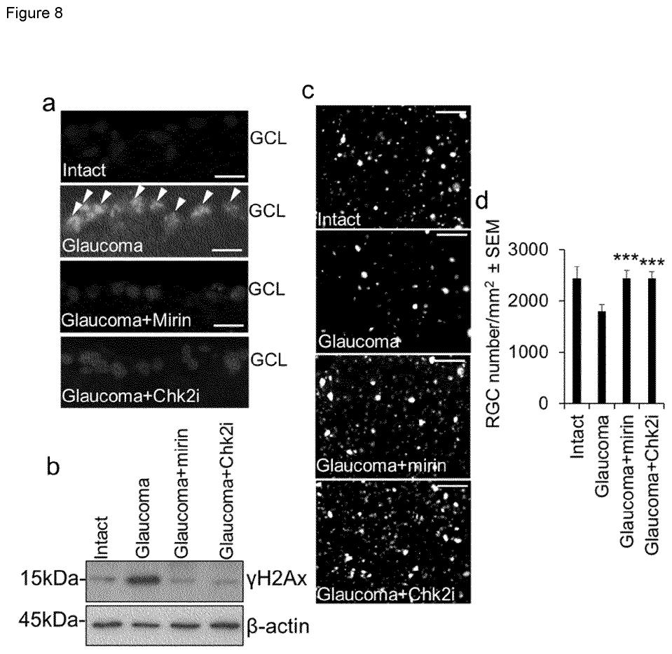

FIG. 8. Treatment with mirin and Chk2i in glaucoma suppresses DSBs in RGC (arrowheads) and promotes RGC survival. GCL=ganglion cell layer. a immunohistochemistry for γH2Ax (red; Blue=DAPI nuclei) in sections of retina at 30 days after induction of glaucoma with intracameral injections TGFβ1. b Western blot of total retinal protein confirms high levels of γH2Ax after induction of glaucoma whilst treatment with mirin and Chk2i suppresses these levels. β-actin is used as a loading control. Scale bars in (A)=scale bars in (B)=100 μm. c Retina wholemounts and d quantification shows enhanced RGC survival after mirin and Chk2i. n=18 retinae/treatment. ***P<0.0001, ANOVA;

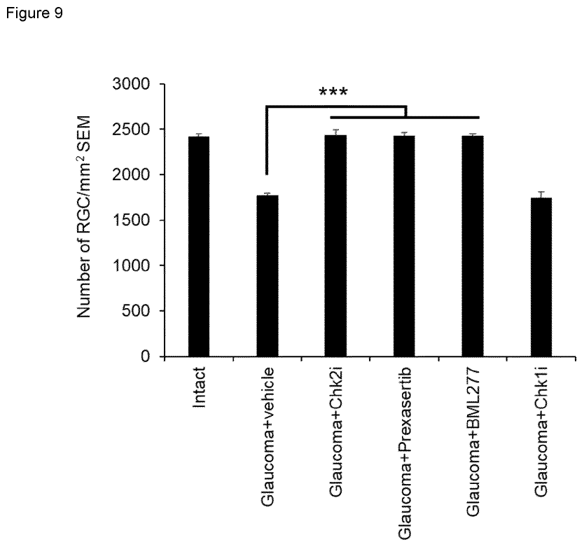

FIG. 9. Comparison of treatment with Chk2 inhibitors in glaucoma models to promote RGC survival. Quantification of retina wholemounts after treatment with CCT241522 (Chk2i), Prexasertib, BML-277 all protect against RGC death induced by glaucoma. Chk1i has no effect on RGC survival n=18 retinae/treatment. ***P<0.0001, ANOVA with Bonferroni's post hoc test.

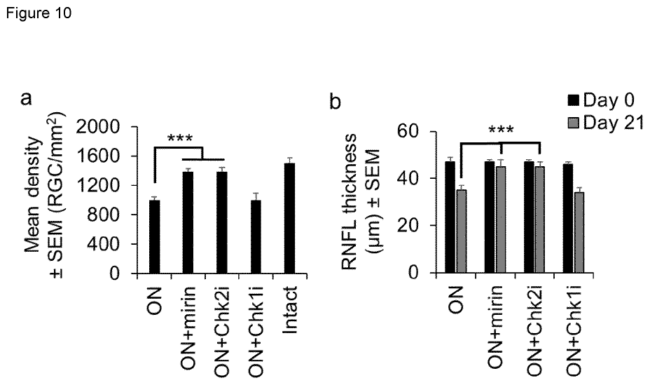

FIG. 10. Inhibition of Mre11 and Chk2 in optic neuritis (ON) promotes RGC survival. a Quantification of Fluorgold backfilled RGC in retinal wholemounts to show that RGC are protected from death by Mre11 and Chk2 inhibitors. b RNFL thickness is preserved in mirin and Chk2i-treated mice. n=18 eyes/treatment; and

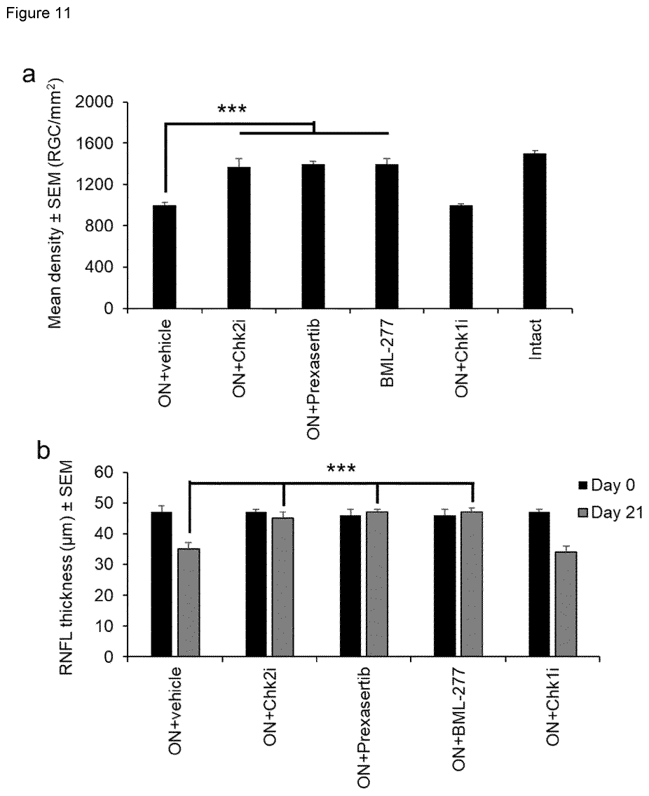

FIG. 11. Inhibition of Chk2 promotes RGC survival in optic neuritis (ON). a Quantification of Fluorgold backfilled RGC in retinal wholemounts to show that RGC are protected from death by CCT24152 (Chk2i), Prexasertib and BML-277. Chk1i has no effect on RGC survival. b RNFL thickness is preserved in Chk2i-, Prexasertib- and BML-277-treated mice. n=18 eyes/treatment. ***=P<0.0001, ANOVA with Bonferroni's post hoc test.

### METHODS

Ethics statement. Experiments were licensed by the UK Home Office and all experimental protocols were approved by the University of Birmingham's Animal Welfare and Ethical Review Board. All animal surgeries were carried out in strict accordance to the guidelines of the UK Animals Scientific Procedures Act, 1986 and the Revised European Directive 1010/63/EU and conformed to the guidelines and recommendation of the use of animals by the Federation of the European Laboratory Animal Science Associations (FELASA). Experiments on the eye and optic nerves also conformed to the ARVO statement for use of animals in research, except that bilateral optic nerve crushes are a condition imposed by the UK Home Office. This is viewed as ‘reduction’ in keeping with the 3R's principle since rats do not use sight as a primary sense and none of the normal behaviours are altered as a result. Adult female Sprague-Dawley rats weighing 170-220 g (Charles River, Margate, UK) were used in all experiments. Animals were randomly assigned to treatment groups with the investigators masked to the treatment conditions. Pre- and post-operative analgesia was provided as standard and as recommended by the named veterinary surgeon.

Drosophila methods. The Drosophila experiments were essentially performed as described in (Taylor and Tuxworth, 2019). Briefly, Tandem Aβ₁₋₄₂ peptides (see Speretta et al (2012) were expressed in adult neurons under the control of Elav-Gal4 with expression suppressed until 7-10 days after eclosion by inclusion of Gal80^{ts}. Flies were maintained at 18° C. to repress expression and shifted to 27° C. to induce expression. Longitudinal tracking of the startle response of flies was performed as in (Taylor and Tuxworth, 2019).

Drosophila strains. Virgin females of the driver line: w¹¹¹⁸, elav-Gal4^{c155}; Gal80^{ts} were used for all crosses. UAS-tAb1-42 12-linker was described in Speretta et al, (2012) and was a kind gift of Dr Damien Crowther. UAS-RNAi lines were obtained from the Bloomington Drosophila Stock Center:
tefu (ATM): TRiP.GL00138 (BL44417)
lok (Chk2): TRiP.GL00020 (BL35152)
mei-41 (ATR): TRiP.GL00284 (BL41934)
grp (Chk1): TRiP.JF2588 (BL27277)

Rat DRGN and retinal cultures. Primary adult rat DRGN and retinal cultures (containing enriched populations RGC) were prepared as described by us previously (Ahmed et al., 2005; Ahmed et al., 2006). Briefly, DRGN or retinal cells were cultured in Neurobasal-A (NBA; Invitrogen, Paisley, UK) at a plating density of either 500/well or 125×10³ cells/well in chamber slides (Beckton Dickinson, Oxford, UK) pre-coated with 100 μg/ml poly-D-lysine (Sigma, Poole, UK), respectively. Positive controls included pre-optimised FGF2 (10 ng/ml [Ahmed, 2005]) and CNTF (10 ng/ml; (Ahmed et al., 2006)) for DRGN and RGC cultures, respectively. Cells were cultured for 4 days in a humidified chamber at 37° C. and 5% CO₂ before being subjected to quantitative RT-PCR or immunocytochemistry, as described below.

Chk inhibitor studies. In preliminary experiments, the optimal concentration of CCT241533 (referred to as Chk2i from herein; 10 μM; Cambridge Bioscience, Cambridge, UK), BML-277 (5 μM; Stratech Scientific, Cambridge, UK) and prexasertib (LY2606368, 10 μM, Cambridge Bioscience, Cambridge, UK) that promoted DRGN/RGC survival and neurite outgrowth was determined. The Chk1 inhibitor LY2603618 (referred to from herein as Chk1i; Tocris, Oxford, UK) had no effect on DRGN/RGC survival at 1-50 μM and hence we used 20 μM, which was shown to induce DNA damage in a variety of human lung cancer cell lines including A549 and H1299 (Wang et al., 2014).

Transfection of DRGN cultures with siRNA/shRNA. ON-TARGETplus rat Chk1 shRNA (siChk1; Cat no. J-094741-09-0002) and Chk2 siRNA (siChk2; Cat no. J-096968-09-0002) were purchased from Dharmacon (Lafayette, CO, USA). Lipofectamine 2000 reagent (Invitrogen) was used to transfect DRGN cultures as described by us previously [Morgan-Warren, 2016]. Briefly, the siRNA and transfection reagent were diluted in NBA (without antibiotics) and allowed to form complexes before beingadded to the cells, and transfected for incubated at 37° C. and 5% CO₂ for 4 days. NBA alone, Lipofectamine alone (Sham) and Liopfectamine+siEGFP (siEGFP) were used as controls. A dose-response assay was undertaken initially, with both siChk1 and siChk2 at 5, 10, 20, 50 and 100 nM concentrations, confirming that a concentration of 10 nM of each optimally knocked down the appropriate mRNA.

Optimal concentrations of each siRNA were then used to determine the effect of Chk1 and Chk2 knockdown on DRGN survival and neurite outgrowth. Immunocytochemistry for βIII-tubulin which marks DRGN soma and neurites was used to quantify survival and neurite outgrowth as described below and by us previously [Ahmed, 2005]. All in vitro experiments consisted of three wells per treatment condition and repeated with cultures from at least three independent animals.

SMARTvector Lentiviral rat Chk1 shRNA (shChk1; Cat no. V3SR11242-239228992) and Chk2 shRNA (shChk2; Cat no. V3SR11242-243372901) driven by a CMV promoter were purchased from Dharmaconand plasmid DNA was prepared according to the manufacturer's instructions. DRGN cultures were transfected with appropriate shRNA using in vivo-jetPEI (Polyplus Transfection, New York, USA) according to the manufacturer's instructions and as described by us previously (Almutiri et al., 2018). DRGN were transfected withplasmid DNA containing control empty vector (shNull; CMV promoter but empty vector), shChk or shChk2. Additional controls included untreated DRGN (NBA) and DRGN transfected with in vivo-jetPEI only (Sham). DRGN were allowed to incubate for 4 days before harvesting of cells and extraction of total RNA for validation of Chk1 and Chk2 mRNA knockdown using quantitative RT-PCR (qRT-PCR), as described below. Immunocytochemistry for βIII-tubulin which marks DRGN soma and neurites was used to quantify survival and neurite outgrowth as described below and by us previously (Ahmed et al., 2005). All in vitro experiments consisted of three wells per treatment condition and repeated with cultures from at least three independent animals.

Immunocytochemistry. Cells were fixed in 4% paraformaldehyde, washed in 3 changes of PBS before being subjected to immunocytochemistry as described by us previously (Ahmed et al., 2005; Ahmed et al., 2006). To visualise neurites, DRGN or RGC were stained with monoclonal anti-βIII tubulin antibodies (Sigma) and detected with Alexa-488 anti-mouse secondary antibodies (Invitrogen). Slides were then viewed with an epi-fluorescent Axioplan 2 microscope, equipped with an AxioCam HRc and running Axiovision Software (all from Carl Zeiss, Hertfordshire, UK). The proportion of DRGN with neurites, the mean neurite length and the number of surviving βIII-tubulin⁺ RGC were calculated using Axiovision Software by an investigator masked to the treatment conditions, as previously described (Ahmed et al., 2005; Ahmed et al., 2006).

DC crush injury model. Rats were injected subcutaneously with 0.05 ml Buprenorphine to provide analgesia prior to surgery and anaesthetised using 5% of Isoflurane in 1.8 ml/l of O₂ with body temperature and heart rate monitored throughout surgery. After partial T8 laminectomy, DC were crushed bilaterally using calibrated watchmaker's forceps [Surey, 2014] and either vehicle, Chk1i, Chk2i, BML-277 or prexasertib, were injected intrathecally. The subarachnoid space was cannulated with a polyethylene tube (PE-10; Beckton Dickinson) through the atlanto-occipital membrane as described by us and others (Tuxworth et al., 2019; Yaksh and Rudy, 1976). Animals were injected immediately with vehicle (PBS), mirin or KU-60019 followed by a 10 μl PBS catheter flush and injections were repeated every 24 hr.

Chk2 inhibition studies in the DC crush injury model. In pilot dose finding experiments, Chk2 inhibition by Chk2i, BML-277 and Prexasertib were all intrathecally injected as described above at 1, 2, 3, 5 and 10 μg (n=3 rats/group, 2 independent repeats) in a final volume of 10 μl saline either daily, every other day or twice weekly for 28 d (Tuxworth et al., 2019). Rats were then killed and L4/L5 DRG on both sides were dissected out, pooled together (n=4 DRG/rat, 12 DRG/group), lysed in ice cold lysis buffer, separated on 12% SDS PAGE gels and subjected to western blot detection of pChk2 levels (Surey et al., 2014). We determined that the amount of Chk2i, BML-277 and prexasertib required to optimally reduce pChk2 levels by intrathecal delivery was 2 μg (final conc=1.37 mM), 3 μg (final conc=451.9 μM) and 3 μg (final conc=547.4 μM), respectively with an optimal dosing frequency of every 24 hrs. The optimal doses of all Chk2 inhibitors was then used for experiments described in this manuscript. Chk1i (LY2603618) was used at equimolar concentrations for each experiment. Rats were killed in a rising concentration of CO₂ at either 28 d for immunohistochemistry and western blot analyses or 6 weeks for electrophysiology and functional tests.

To perform an initial dose response study to knock down Chk2 in vivo after DC injury by shRNA, 1, 2, 3 and 4 μg of plasmid DNA for shNull, shChk1 and shChk2 (all from Dharmacon) were complexed in in vivo-JetPEI and injected intra-DRG as described by us previously (Almutiri et al., 2018). Sham treated animals (partial laminectomy but no DC injury) were also included as additional controls. At 4 weeks after DC injury and treatment, ipsilateral L4/L5 DRG pairs were harvested, total RNA extracted using Trizol reagent as described above and knock down of Chk1 and Chk2 mRNA knockdown using quantitative qRT-PCR, as described above. Contralateral L4/L5 DRG pairs were treated the same as above and used as controls. In further experiments, the optimal dose of 2 μg of each respective shRNA was used. This included western blot to determine pChk1 and pChk2 levels after shChk2 treatment. For these experiments, animals were randomly assigned to DC+shNull and DC+shChk2 groups each comprising n=6 rats and repeated on 3 independent occasions (total n=18 rats/group). Ipsilateral L4/L5 DRG pairs were harvested at 4 weeks after DC injury and treatment and total protein extracted, subjected to western blot and probed for pChk1 and pChk2 to determine pChk2 suppression after shChk2-mediated knockdown of Chk2 mRNA. Finally, to determine if Chk2 suppression by shChk2 also promotes similar levels of electrophysiological, sensory and locomotor improvements as Chk2i, n=6 rats/group (3 independent repeats (total n=18 rats/group)) animals were randomly assigned to Sham, shNull, shChk1 and shChk2 groups. Animals received intra-DRG injections of shNull, shChk1 and shChk2 immediately after DC injury, as described by us previously (Almutiri et al., 2018). Animals were allowed to survive for 6 weeks with functional testing (tape sensing+removal and ladder crossing tests) performed pre- and post-DC injury as described below. Electrophysiology was performed on the same set of animals at 6 weeks after DC injury and treatment as described below.

Optic nerve crush injury (ONC) model. Optic nerves were crushed bilaterally 2 mm from the globe of the eye as described previously (Berry et al., 1996). In pilot dose-finding experiments, Chk2i was intravitreally injected at 1, 2, 3, 5, and 10 μg (n=3 rats/group, 2 independent repeats), without damaging the lens, immediately after ONC. To determine optimal doses and dosing frequency, Chk2i was injected every other day, or twice weekly or once every 7 days, in a final volume of 5 μl saline for 24 days. Rats were then killed and retinae were dissected out, lysed in ice-cold lysis buffer, separated on 12% SDS PAGE gels and subjected to western blot detection of pChk2 levels (not shown). We determined that the dosing frequency of twice weekly and 5 μg of Chk2i optimally reduced pChk2 levels. Chk1i was used at the same dose as Chk2i. Optimal doses were then used for all experiments described in this manuscript. Rats were killed in rising concentrations of CO₂ at 24 days after ONC injury for western blot analyses or for determination of RGC survival and axon regeneration, as described below. For the experiments reported in this manuscript, n=6 rats/group were used and assigned to: (1), Intact controls (no surgery to detect baseline parameters); (2), ONC+vehicle (ONC followed by intravitreal injection of vehicle solution); (3), ONC+Chk1i (ONC followed by intravitreal injection of equimolar concentration of Chk1i, twice weekly); and (4), ONC+Chk2i (ONC followed by intravitreal injection of 5 μg of Chk2i). Each experiment was repeated on 3 independent occasions with a total n=18 rats/group/test.

Induction of glaucoma. Glaucoma was induced in adult rat Sprague-Dawley rats using a TGFβ2 model that causes scarring in the trabecular meshwork and hence raises intraocular pressure, as described by us previously (Hill et al., 2015). At day zero, a self-sealing incision was made though the cornea into the anterior chamber enabling twice weekly intracameral injections of 3.5 μl of TGFβ2 (5 ng/μl) using glass micropipettes for 30 days. Vehicle, comprising 0.9% saline, was injected in control groups. Intraocular pressure was measured using an iCare

Tonolab rebound tonometer (Icare, Helsinki, Finland). By 7 days, the intraocular pressure begins to rise and is sustained for the duration of the experiment.

Induction of optic neuritis. Optic neuritis was induced in transgenic MOG^{TCR}xThy1CFP mice as described by us previously (Lidster et al., 2013). Animals were intraperitoneally injected with 150 ng Bordetella pertussis toxin on day 0 and 2. Animals were monitored daily and assessed for the development of EAE. At the end of the experiment, animals were then killed by CO₂ overdose.

Measurement of RNFL thinning using optical coherence tomography (OCT). A Spectralis HRA+OCT machine was used to capture OCT images. Examinations were recorded in both right (oculus dextrus, OD) and left eyes (oculus sinister, OS) of each animal at day 0 and day 21 after immunisation. To capture an OCT image, animals were anaesthetised and placed on the animal mount and an infra-red (IR) reflection image with the optic nerve head in a centralised position was achieved with optimal focus (approx+18.0 dioptres). A RNFL single exam using the automatic real time (ART) mode (allows averaging of 100 recordings) was produced for each mouse eye, which automatically measured RNFL thickness (μm) in a 30° circle surrounding the optic nerve head.

### Assessment of RGC Survival

FluoroGold backfilled RGC in retinal wholemounts were used to determine RGC survival as described previously (Berry et al., 1996). Briefly, at 22 days after ONC, 4% FluoroGold (FG; Cambridge Bioscience, Cambridge, UK) was injected into the ON, between the lamina 15 cribrosa and the optic nerve crush site, retinae dissected out, flattened onto charged glass microscope slidesphotographed and the number of FG-labelled RGC were then counted blind using ImagePro Version 6.0 (Media Cybernetics) from captured images of 12 rectangular areas (0.36×0.24 mm)/retinae and the number of RGC/mm² was calculated, as described by us previously (Ahmed et al., 2011).

Immunohistochemistry. Tissue preparation for cryostat sectioning and immunohistochemistry were performed as described by us previously (Surey et al., 2014). Briefly, rats were intracardially perfused with 4% formaldehyde and L4/L5 DRG and segments of T8 cord containing the DC injury sites and optic nerves were dissected out and post-fixed for 2 h at room temperature. Tissues were then cryoprotected in a sucrose gradient prior to mounting in optimal cutting temperature (OCT) embedding medium (Raymond A Lamb, Peterborough, UK) and frozen on dry ice. Samples were then sectioned using a cryostat and immunohistochemistry was performed on sections from the middle of the DRG or optic nerve as described previously (Surey et al., 2014; Ahmed et al., 2006). Sections stained with mouse anti-γH2Ax (1:400 dilution; Merck Millipore, Watford, UK), rabbit anti-NF200 (1:400 dilution;

Sigma, Poole, UK) and mouse anti-GAP43 (1:400 dilution; Invitrogen, Poole, UK) primary antibodies overnight at 4° C. After washing in PBS, sections were incubated with Alexa-488 anti-mouse and Texas red anti-rabbit IgG secondary antibodies, for 1 h at room temperature prior to further washes in PBS and mounting in Vectashield containing DAPI (Vector Laboratories, Peterborough, UK). Controls were included in each run where the primary antibodies were omitted and these sections were used to set the background threshold prior to image capture. Sections were viewed using Axioplan 200 an epi-fluorescent microscope equipped with an Axiocam HRc and running Axiovision Software (all from Zeiss, Herefordshire, UK). Image capture and analysis was performed by an investigator masked to the treatment conditions.

Quantification of DC axon regeneration. GAP43⁺ axons were quantified according to previously published methods (Hata et al., 2006). Briefly, the number of intersections of GAP43⁺ fibers were counted through a dorsoventral orientated line in reconstructed serial parasagittal sections of the cord (serial 50μm-thick sections ˜70-80 sections/animal; n=10 rats/treatment)). Axon number was then represented as % of fibers counted at 4 mm above the lesion, where the DC was intact.

### Quantification of RGC Axon Regeneration

The number of regenerating GAP43+RGC axons were counted at ×400 magnification in ON sections after drawing a vertical line through the axons and counting the number of axons extending beyond this line, using previously published methods (Vigneswara et al., 2013).

Protein extraction and western blot analysis. Total protein from ipsilateral L4/L5 DRG was extracted and subjected to western blot followed by densitometry according to our previously published methods (Ahmed et al., 2005; Ahmed et al, 2006). Briefly, 40 μg of total protein extract was resolved on 12% SDS gels, transferred to polyvinylidene fluoride (PVDF) membranes (Millipore, Watford, UK) and probed with relevant primary antibodies: anti-pChk1/pChk2 (both used at 1:200 dilution, Cell Signalling Technology, Danvers, CA, USA). Monoclonal β-actin (1:1000 dilution, Sigma) was used as a loading control. Membranes were then incubated with relevant HRP-labelled secondary antibodies and bands were detected using the enhanced chemiluminescence kit (GE Healthcare, Buckinghamshire, UK). For densitometry, western blots were scanned into Adobe Photoshop (Adobe Systems Inc, San Jose, CA, USA) and analysed using the built-in-macros for gel analysis in ImageJ (NIH, USA, http://imagej.nih.gov/ij).

### Electroretinography (ERG)

mERG were recorded (HMsERG—Ocuscience, Kansas City, MO, USA) at 24 days post injury and in uninjured controls and were interpreted using ERG View (Ocuscience) (Blanch et al., 2012). Briefly, animals were dark-adapted (scotopic) overnight and flash ERG were recorded from −2.5 to +1 log units with respect to standard flash in half log unit steps and photopic (light-adapted) flash ERG were recorded with background illumination of 30,000 mcd/m2 over the same range. ERG traces were analysed using ERG View (Ocuscience) and marker position manually verified and adjusted where necessary by an observer masked to the treatment conditions.

Electrophysiology. Six weeks after surgery or treatment, compound action potentials (CAP) were recorded after vehicle, Ck2i, Chk1i, BML-277 and prexasertib treatment as previously described (Almutiri et al., 2018). Briefly, with the experimenter masked to the treatment conditions, silver wire electrodes applied single-current pulses (0.05 ms) through a stimulus isolation unit in increments (0.2, 0.3, 0.6, 0.8, and 1.2 mA) at L1-L2 and compound action potentials (CAP) were recorded at C4-C5 along the surface of the midline spinal cord. Spike 2 software was then used to calculate CAP amplitudes between the negative deflection after the stimulus artifact and the next peak of the wave. CAP area was calculated by rectifying the negative CAP component (full-wave rectification) and measuring its area. At the different stimulation intensities. The dorsal half of the spinal cord was transected between the stimulating and recording electrodes at the end of the experiment to confirm that a CAP could not be detected. Representative CAP traces are processed output data from Spike 2 software.

Functional tests. Functional testing after DC lesions was carried out as described previously (Almutiri et al., 2018; Tuxworth et al., 2019). Briefly, animals (n=6 rats/group, 3 independent repeats; total n=18/group) received training to master traversing the horizontal ladder for 1w before functional testing. Baseline parameters for all functional tests were established 2-3 d before injury. Animals were then tested 2 d after DC lesion+treatment and then weekly for 6 weeks. Experiments were performed by 2 observers (treatment conditions were masked) in the same order, the same time of day and each test performed for 3 individual trials. Horizontal ladder test: This tests the animals locomotor function and is performed on a 0.9-meter-long horizontal ladder with a diameter of 15.5 cm and randomly adjusted rungs with variable gaps of 3.5-5.0 cm. The total number of steps taken to cross the ladder and the left and right rear paw slips being were recorded and the mean error rate was then calculated by dividing the number of slips by the total number of steps taken.

Tape sensing and removal test (sensory function): The tape sensing and removal test determines touch perception from the left hind paw. Animals were held with both hind-paws extended and the time it took for the animal to detect and remove a 15×15 mm piece of tape (Kip Hochkrepp, Bocholt, Germany) was recorded and used to calculate the mean sensing time.

Statistical analysis. Data are presented as means±SEM. When data were normally distributed, significant differences were calculated using SPSS Version 22 (IBM, NJ, USA) software by one-way analysis of variance (ANOVA), with Bonferroni post hoc tests, set at P<0.05.

For the horizontal ladder crossing functional tests, data was analysed using R package (www.r-project.org) and whole time-course of lesioned and sham-treated animals were compared using binomial generalized linear mixed models (GLMM) as described previously [Fagoe, 2016; Tuxworth, 2019]. Thus, data was compared using binomial GLMM, with lesioned/sham (‘LESION’; set to true in lesioned animals post-surgery, false otherwise) and operated/unoperated (‘OPERATED’; set to false before surgery, true after surgery) as fixed factors, animals as a random factors and time as a continuous covariate. Binomial GLMMs were then fitted in R using package Ime4 with the glmer functions and P values calculated using parametric bootstrap.

For the tape sensing and removal test, linear mixed models (LMM) were calculated by model comparison in R using the package pbkrtest, with the Kenward-Roger method [Fagoe, 2016; Almutiri, 2018]. Independent sample T-tests were performed to determine statistical differences at individual time points.

ATM and ATR mediate many of the downstream events such as cell-cycle arrest, repair and apoptosis through activation of either checkpoint kinase-2 (Chk2) or checkpoint kinase-1 (Chk1), respectively¹⁰.

If persistent activation of the DNA damage pathway causes neuronal dysfunction and potentially apoptosis, then suppression of the ATM-Chk2 or ATR-Chk1 pathways may be protective to neurons and help preserve function. However, knowing where to target each pathway is unknown. We tested this in an adult-onset paradigm of chronic amyloid toxicity in Drosophila where DSBs form in neurons^{11,12} and observed a clear protective effect by knocking down expression of ATM in the Aβ₁₋₄₂-expressing adult neurons (FIG. 1a). Surpisingly, knockdown of Chk2, a key downstream protein of ATM was also protective (FIG. 1b). ATR is primarily activated during DSB repair by homologous recombination, which requires a sister chromatid as template and is not likely to be available to post-mitotic neurons. However, knockdown of ATR or its downstream target, Chk1, were also protective (FIG. 1c,d). but knockdown of a regulator of single-strand break repair, PARP-1, had no effect (FIG. 1e). Consistent with a protective effect, the lifespan of Aβ₁₋₄₂ -expressing flies was significantly extended by knockdown of ATM, Chk2, ATR or Chk1 (FIG. 3).

We asked whether inhibiting Chk1 and Chk2 activity would be neuroprotective in models of spinal cord injury (SCI) and optic nerve injury^{15,16}. In primary adult rat dorsal root ganglion neuron (DRGN) cultures, Chk2 was phosphorylated at the ATM target residue, Thr68, and at an autophosphorylation site, Thr383, required for activation (FIG. 1f,g). Treatment with the specific Chk2 inhibitor, CCT241533 (termed Chk2i herein), suppressed Chk2 phosphorylation (FIG. 1f,g) and improved DRGN survival from 40% in NBA-treated controls to 90% in Chk2i-treated wells (FIG. 1h,i). Chk2i also stimulated neurite outgrowth in DRGN over and above that observed for the positive control, FGF2 42% to 82%) (FIG. 1j), and those neurites were significantly longer when compared to controls (12 μm to 520 μm) (FIG. 1k) or FGF2 treatment (180 μm to 520 μm) (FIG. 1k). In contrast, treatment with the Chk1 inhibitor, LY2603618, (termed Chk1i herein) had no effect on DRGN survival or neurite outgrowth (FIG. 1j,k).

We extended our findings to ask if suppression of Chk2 activity promotes axon regeneration and functional recovery in vivo using the translationally relevant model of T8 dorsal column (DC) crush model of spinal cord injury (SCI) in rats ^{15,17} Chk2 was phosphorylated at both Thr68 and Thr383 at 28 days after injury but this was abolished by daily intrathecal injections of Chk2i (FIG. 2a,b). No changes in Chk1 phosphorylation was induced by DC injury or by Chk2i treatment (FIG. 2a,b). Chk2i promoted significant DC axon regeneration at all distances rostral to the lesion site despite the presence of spinal cord cavities, with 23.7% of the axons regenerating 6 mm rostral to the lesion site (FIG. 2c,d). In contrast, Chk1i and vehicle-treated rats showed no axon regeneration beyond the lesion site (FIG. 2c,d).

Electrophysiological recordings demonstrated that Chk2i significantly improved the negative CAP trace across the lesion site (FIG. 2e), increased the CAP amplitude at all stimulation intensities (FIG. 2f) and improved the CAP area to within 20% of that observed for sham-treated control groups and >90% when compared to the vehicle or Chk1i treatments (FIG. 2g). Animal performance in the tape sensing/removal test for sensory function (FIG. 2h) and the ladder crossing test for locomotor function (FIG. 2i) both showed significant improvements after only 2 days of Chk2i treatment compared to vehicle or Chk1i treatment. Remarkably, 3 weeks after injury sensory (FIG. 2h) and locomotor (FIG. 2i) performance were both indistinguishable from sham-treated animals. These improvements in electrophysiological, sensory and locomotor function were confirmed in vivo by treatment with a variety of different Chk2 inhibiotrs. BML-277, a potent Chk2 inhibitor with an IC₅₀ of 15 nM (FIG. 4), an shRNA to Chk2 (shChk2) to knock down Chk2 mRNA/protein (FIG. 5) and prexasertib, a Chk1/Chk2 inhibitor with an IC₅₀ of 8 nM for Chk2 and has been through to Phase 2 clinical trials ¹⁸ (FIG. 6).

Finally, we asked if Chk2 inhibition can be neuroprotective in a second in vitro and in vivo model of CNS acute trauma: the optic nerve crush (ONC) injury model ^{16,19} Again, Chk2 but not Chk1 inhibition promoted significant RGC survival and neurite outgrowth in vitro (FIG. 6a-d) and intraocular delivery of Chk2i to ONC-injured rats promoted >90% RGC survival and significant RGC axon regeneration (FIG. 6e-h) accompanied by significant (>83%) improvement in RGC function measured by flash electroretinography (ERG) amplitude (FIG. 6i,j).

The use of Chk1/Chk2 inhibitors, such as prexasertib or nucleic acid based Chk2 inhibition, such as AAV-mediated Chk2 knockdown are an exciting new approach with potential to address the unmet clinical needs of neurotrauma patients. Inhibition of Chk2 activity in two translationally-relevant models of acute neurotrauma produces a far greater neuroprotective and neuroregenerative effect than any previously identified treatment 20-22. Moreover, the methods of delivery—intrathecal for SCI or intraocular for ONC—are directly translatable to neurotrauma patients.

We then asked the if inhibition of Chk2 is also neuroprotective in eye diseases where RGC death occurs. In glaucoma, the death of approximately 30% of RGC occurs over time [Hill et al., 2015; PMID: 26066743]. We demonstrated by immunohistochemistry (FIG. 8a) and western blot (FIG. 8b) that significant immunoreactivity was present for γH2Ax, indicative of DNA damage. However, treatment with either mirin which inhibits MRE11, or Chk2i attenuates the levels of γH2Ax (FIG. 8a and b) and protects >98% of RGC from death at 30 days after the induction of glaucoma (FIG. 8c and d). All of the Chk2 inhibitors tested, including BML-277, CCT245133 and Prexasertib all promoted >98% protection of RGC from death, whilst Chk1i had no effect of RGC survival (FIG. 9).

In a second model of disease-related RGC death, the optic neuritis model, where 30% of RGC death occurs over a period of 21 days after induction [Lidster et al., 2014), we also asked the question if Chk2 inhibitors had beneficial effects on RGC neuroprotection. Inhibition of MRE11 with mirin and Chk2 with Chk2i protected >96% of RGC from death (FIGS. 10a) and >97% protection against retinal nerve fibre layer thinning (FIG. 10b), in this disease model. Treatment with BML-277, Chk2i and Prexasertib all protected >96% of RGC from death and >97% protection against retinal nerve fibre layer thinning, whilst treatment with Chk1i had no effect (FIG. 11).

Taken together, these results show that inhibition of Chk2 and not Chk1 protects against loss of function in SCI models and protects from RGC death after optic nerve injury and diseases where RGC death occurs.

### REFERENCES

Simpson J. E., et al. Neuropathol Appl Neurobiol 41: 483-496 (2015).
Merlo D., et al. Neural Plast 2016: 1206840 (2016).
Nagy Z., et al. Acta Neuropathol 94: 6-15 (1997).
Lamarche B. J., et al. FEBS Lett 584: 3682-3695 (2010).
Taylor M. J. & Tuxworth R. I. J Neurogenet 33: 190-198 (2019).
Tuxworth R. I. T., et al. Brain Comms 1: fcz005 (2019).
Surey S., et al. Neuroscience 275: 62-80 (2014).
Ahmed, Z., et al. Brain 129: 1517-1533 (2006).
Almutiri S., et al. Sci Rep 8: 10707 (2018).
Lee J. M., et al. Lancet Oncol 19: 207-215 (2018).
Vigneswara V. & Ahmed Z. Cell Death Dis 10: 102 (2019).
Ahmed Z., et al. Cell Death Dis 2: e173 (2011).
de Lima, S., et al. Proc Natl Acad Sci USA 109: 9149-9154 (2012).
Pernet V., et al. Neurobiol Dis 51: 202-213 (2013).
Joung J. K., & Sander J. D. Nat Rev Mol Cell Biol 14: 49-55 (2013).
Pickar-Oliver A. & Gersbach C. A. Nat Rev Mol Cell Biol 20: 490-507 (2019).
Setten M. M., et al. Nat Rev Dru Discov 19: 695-710 (2019).
Jobson A. G., et al. J Pharmacol Exp Ther 331: 816-826 (2009).
Zabludoff S., et al. Mol Cancer Ther 7: 2955-66 (2008).
Anderson V. E., et al. Cancer Res 71: 463-472 (2011). Arienti K. L., et al. J Med Chem 48: 1873-85 (2005).
King C. et al. Mol Cancer Ther 14: 2004-13 (2015).
Kitchen P., et al. Cell 181: 784-799 (2020).
Speretta E. et al. J Biol Chem 287: 20748-54(2012).
Wang T., et al. Science 343: 80-84 (2014).
Morgan-Warren P. J., et al. Invest Ophthalmol Vis Sci 57: 429-443 (2016).
Yaksh T. L., and Rudy T, A. Physiol Behav 17: 1031-1036 (1976)
Lidster et al., PLoS ONE 8:e79188 (2013).
Neumann, S and Woolf C. J. Neuron 23: 83-91 (1999).
Neumann, S., et al., Neuron 34: 885-893 (2002).
Blanch R J., et al. Invest Ophthalmol Vis Sci 53: 7220-7226 (2012).
Hata K., et al. J Cell Biol 173: 47-58 (2006).
Hill, L. J., et al. Invest Ophthalmol Vis Sci 56: 3743-3757 (2015).
Khalil H. S., et al, BioDiscovery 1: e8920 (2012)
Ahmed Z., et al., Neurobiol Dis 37: 441-454 (2010).

1. A Chk2 inhibitor for use in a method of preventing or treating an ocular condition.

2. The Chk2 inhibitor for use according to claim 1, wherein the ocular condition is associated with neuronal damage/dysfunction in the eye, or neurons in communication with the eye.

3. The Chk2 inhibitor for use according to claim 1 or 2, wherein the Chk2 kinase inhibitor has a neuroprotective and/or neuroregenerative effect.

4. The Chk2 inhibitor for use according to claims 1 to 3, wherein the subject has or is at risk of developing an ocular condition.

5. The Chk2 inhibitor for use according to any one claims 2 to 4, wherein the neuronal damage/dysfunction or neuronal degeneration is caused by, or may result from, physical trauma, chemical means, infection, inflammation, hypoxia and/or interruption in blood supply.

6. The Chk2 inhibitor for use according to claim 5, wherein the method comprises administering the Chk2 inhibitor to the subject prior to surgery or administration of a drug, and/or after surgery or administration of a drug.

7. The Chk2 inhibitor for use according to claim 4, wherein the ocular condition is a neurodegenerative disorder and/or autoimmune disease.

8. The Chk2 inhibitor for use according to any of claims 1 to 7, wherein the neuronal damage/dysfunction or neurological condition is due to physical trauma, caused by a subject receiving physical damage to the eye or surrounding tissue due to an external force, or material penetrating the tissue, or physical trauma to the head in general, which can further lead to associated problems in the eye.

9. The Chk2 inhibitor for use according to any one of the preceding claims, wherein the Chk2 inhibitor inhibits expression or activity of Chk2.

10. The Chk2 inhibitor for use according to any one of the preceding claims, wherein the Chk2 inhibitor is a small molecule, protein, peptide or nucleic acid.

11. The Chk2 inhibitor for use according any of claims 1-9 wherein the Chk2 inhibitor is a protein, peptide, antibody or antibody fragment which is capable of specifically binding Chk2 and inhibiting its activity.

12. The Chk2 inhibitor for use according to claim 10 wherein the Chk2 inhibitor is PV1019, AZD7762, CCT241533, BML-277 or prexasertib.

13. The Chk2 inhibitor for use according to claim 6 wherein the trauma is associated with retinal ischemia, acute retinopathy associated with trauma, postoperative complications, traumatic optic neuropathy (TON); and damage related to laser therapy (including photodynamic therapy (PDT)), damage related to surgical light-induced iatrogenic retinopathy, or damage related to corneal transplantation and stem cell transplantation of ocular cells.

14. The Chk2 inhibitor for use according to claim 7 wherein the ocular condition is optic neuritis, multiple sclerosis (MS), glaucoma, or a neurodegenerative condition where damage to neurons within the eye is an associated or secondary issue, such as Parkinson's disease; Alzheimer's disease; amyotrophic lateral sclerosis (ALS); Huntington's disease, neuronal ceroid lipofuscinoses (NCLs) or a related lysosomal storage disorder, where progressive optic atrophy occurs.

15. An ophthalmic formulation comprising a Chk2 inhibitor, together with an ophthalmically acceptable excipient therefor.

16. The ophthalmic formulation according to claim 16 to be administered as an irrigating solution, topically, perioclarly, or intraocularly.
